(19) United States
(12) Patent Application Publication
(10) Pub. No.: US 2013/0131764 A1
(43) Pub. Date: May 23, 2013

(54) COOL FAT BURNER

(71) Applicant: P. Eric Grove, Hustontown, PA (US)

(72) Inventor: P. Eric Grove, Hustontown, PA (US)

(21) Appl. No.: 13/673,805

(22) Filed: Nov. 9, 2012

Related U.S. Application Data
(60) Provisional application No. 61/561,346, filed on Nov. 18, 2011.

(51) Int. Cl. A61F 7/10

(52) U.S. Cl. CPC A61F 7/10
USPC 607/108

(57) ABSTRACT

An ice harness for cooling brown adipose tissue. The ice harness includes a front section, wherein the front section is configured to be placed on a user's chest. The ice harness also includes a rear section, wherein the rear section is configured to be placed on the user's upper back. The ice harness further includes a first cold-gel pack, wherein the first cold-gel pack is attached to the front section. The ice harness additionally includes a second cold-gel pack, wherein the second cold-gel pack is attached to the rear section.

### CROSS-REFERENCE TO RELATED APPLICATIONS

This application claims the benefit of and priority to U.S. Provisional Patent Application Ser. No. 61/561,346 filed on Nov. 18, 2012, which application is incorporated herein by reference in its entirety.

### BACKGROUND OF THE INVENTION

Weight loss products are a large yet growing market. However, weight loss methods vary greatly. For example, some weight loss methods are complicated and include facets of the user's whole life. E.g., it may include life style changes, such as changes to the user's diet, exercise regimen and many other changes. These complications tend to discourage potential customers because of the time and stress that may be involved.

In addition, there is much conflicting information. For example, some foods are regarded as foods that assist in weight loss by some and as foods that should be avoided by others. Even classes of foods, such as proteins, carbohydrates, and fats, are often claimed to be both beneficial and detrimental. This means that those who wish to lose weight are often confused about which changes will be beneficial and, therefore, simply fail to make changes to their lifestyle which would result in weight loss.

What is clear, however, is that using the body's own mechanisms are often the most effective at helping one to lose weight. For example, cooling of brown adipose tissue can allow a user to burn white fat, more commonly known as cellulite. In particular, cooling the brown adipose tissue appears to raise the metabolism of the tissue, causing it to deplete a body's reserves of white fat. Thus, cooling brown adipose tissue can allow a user to induce weight loss.

In order to cool brown adipose tissue, some people take ice-baths or cold showers. This is impractical for most people as an ice-bath requires specialized devices and/or large amounts of ice and cold showers may not sufficiently cool the brown adipose tissue. In addition, they are also very uncomfortable for most people. Some university tests involving BAT had subjects sit in excessively cold rooms for an hour or so or had subjects sit with their feet in ice buckets. This is also impractical for most people.

Accordingly, there is a need in the art for a device which allows a user to lose weight without drastic lifestyle changes. Further there is a need in the art for a device which can cool brown adipose tissue. In addition, there is a need in the art for the device to be reusable. Further, there is a need in the art for the device to be adjustable so that it can be worn by multiple users. Moreover, there is a need in the art for the device to allow the wearer to move about.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes an ice harness for cooling brown adipose tissue. The ice harness includes a front section, wherein the front section is configured to be placed on a user's chest. The ice harness also includes a rear section, wherein the rear section is configured to be placed on the user's upper back. The ice harness further includes a first cold-gel pack, wherein the first cold-gel pack is attached to the front section. The ice harness additionally includes a second cold-gel pack, wherein the second cold-gel pack is attached to the rear section.

Another example embodiment includes an ice harness for cooling brown adipose tissue. The ice harness includes a front section, wherein the front section is configured to be placed on a user's chest. The ice harness also includes a rear section, wherein the rear section is configured to be placed on the user's upper back. The ice harness further includes a first cold-gel pack, wherein the first cold-gel pack is attached to the front section. The ice harness additionally includes a second cold-gel pack, wherein the second cold-gel pack is attached to the rear section. The ice harness also includes a shoulder support, wherein the shoulder support is configured to secure the front section and the rear section vertically relative to the user. The ice harness further includes an under-arm support, wherein the under-arm support is configured to secure the front section and the rear section horizontally relative to the user

Another example embodiment includes a method for cooling brown adipose tissue. The method includes cooling a cold-gel pack. The method also includes placing the cold-gel in an ice harness. The ice harness includes a front section, wherein the front section is configured to be placed on a user's chest. The ice harness also includes a rear section, wherein the rear section is configured to be placed on the user's upper back.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A illustrates a front view of the ice harness for cooling brown adipose tissue;

FIG. 1B illustrates a rear view of the ice harness for cooling brown adipose tissue;

FIG. 1C illustrates a side view of the ice harness for cooling brown adipose tissue;

FIG. 1D illustrates a top view of the ice harness for cooling brown adipose tissue;

FIG. 2A is an “inside” view of the ice harness;

FIG. 2B is an “outside” view of the ice harness;

FIG. 3 illustrates an exploded view of the ice harness with attach cold-gel packs; and

FIG. 4 is a flow chart illustrating a method of cooling brown adipose tissue.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

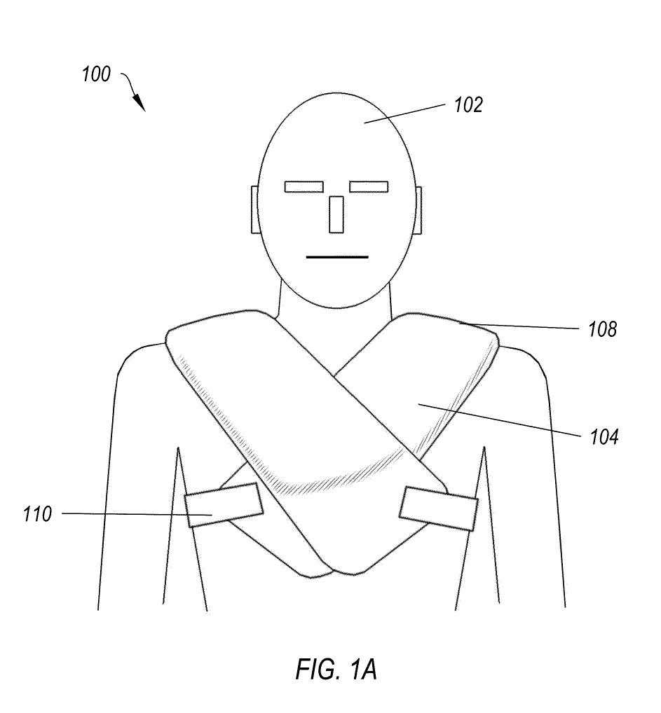

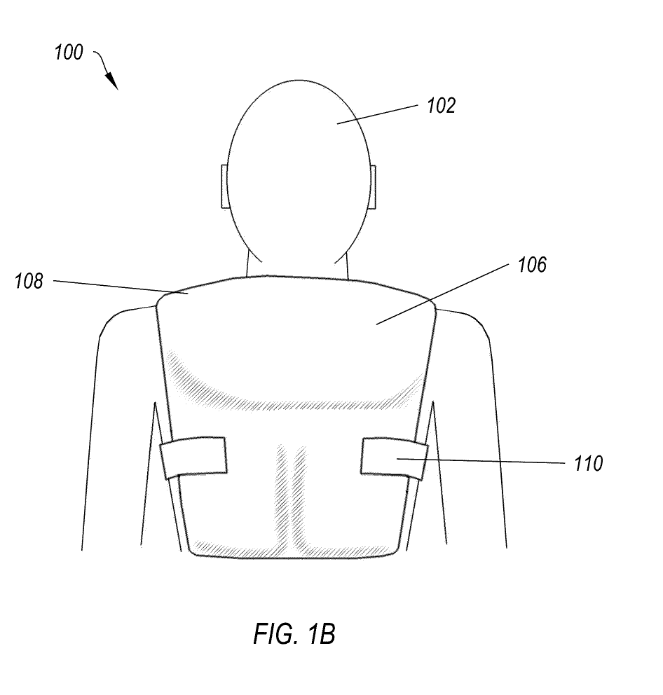

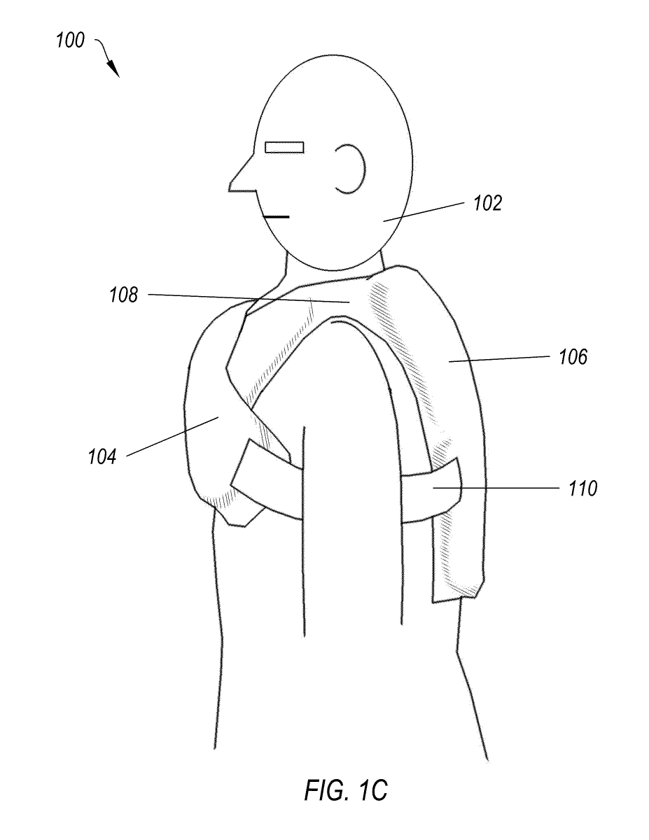

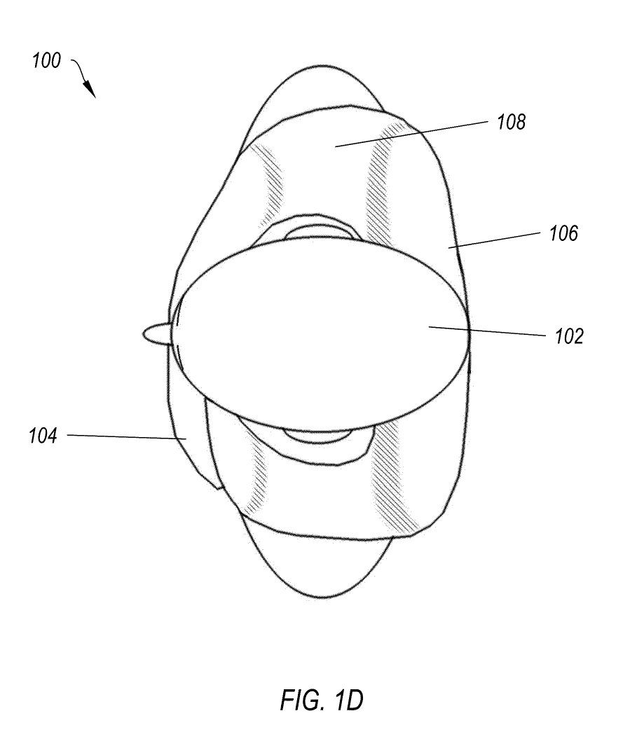

FIGS. 1A, 1B, 1C and 1D illustrate an example of an ice harness 100 for cooling brown adipose tissue (“BAT” or “brown fat”). FIG. 1A illustrates a front view of the ice harness 100 for cooling brown adipose tissue; FIG. 1B illustrates a rear view of the ice harness 100 for cooling brown adipose tissue; FIG. 1C illustrates a side view of the ice harness 100 for cooling brown adipose tissue; and FIG. 1D illustrates a top view of the ice harness 100 for cooling brown adipose tissue. Cooling brown adipose tissue can help a user 102 to lose weight. In addition, cooling brown adipose tissue can increase adiponectin levels. Adiponectin is involved in regulating glucose levels as well as fatty acid breakdown.

In particular, cooling of the brown adipose tissue will cause the brown adipose tissue to burn white fat (i.e., cellulite). That is, brown adipose tissue, when cooled, will burn white fat. The ice harness 100 is designed to specifically target areas that will directly chill and activate BAT, primarily around the collar bones, lower neck, sternum, trapezius, and middle of upper back. Thus, the user can burn cellulite simply by wearing the ice harness 100.

FIGS. 1A, 1B, 1C and 1D show that the ice harness 100 can include a front section 104. The front section 104 is configured to be placed near the user's 102 chest. In particular, the front section 104 can be configured to cool brown adipose tissue in the user's 102 chest. For example, the front section 104 can include one or more pockets for receiving cold-gel packs. Additionally or alternatively, the front section 104 can be made of freezable material or have cold-gel packs attached.

FIGS. 1A, 1B, 1C and 1D also show that the ice harness 100 can include a rear section 106. The rear section 106 is configured to be placed near the user's 102 back. In particular, the rear section 106 can be configured to cool brown adipose tissue in the user's 102 back. For example, the rear section 106 can include one or more pockets for receiving cold-gel packs. Additionally or alternatively, the rear section 106 can be made of freezable material or have cold-gel packs attached.

FIGS. 1A, 1B, 1C and 1D further show that the ice harness 100 can include a shoulder support 108. The shoulder support 108 can support the ice harness 100 relative to the user 102. In particular, the shoulder support 108 can ensure that the front section 104 and the rear section 106 remain in a constant vertical position relative to the user 102. The shoulder support 108 can include any suitable attachment. For example, the shoulder support 108 can include molded plastic or fabric straps. Additionally or alternatively, the shoulder support 108 can attach to the first section 104 and the rear section 106 using any desired method. For example, the shoulder support can attach to the first section 104 and the rear section 106 using hook and loop fasteners, buttons, zippers, snaps or any other desired attachment.

FIGS. 1A, 1B, 1C and 1D additionally show that the ice harness 100 can include an under-arm support 110. In at least one implementation, the under-arm support 110 can support the ice harness 100 relative to the user 102. In particular, the under-arm support 110 can ensure that the front section 104 and the rear section 106 remain in a constant horizontal position relative to the user 102. The under-arm support 110 can include any suitable attachment. For example, the under-arm support 110 can include molded plastic or fabric straps. Additionally or alternatively, the under-arm support 110 can attach to the first section 104 and the rear section 106 using any desired method. For example, the shoulder support can attach to the first section 104 and the rear section 106 using hook and loop fasteners, buttons, zippers, snaps or any other desired attachment.

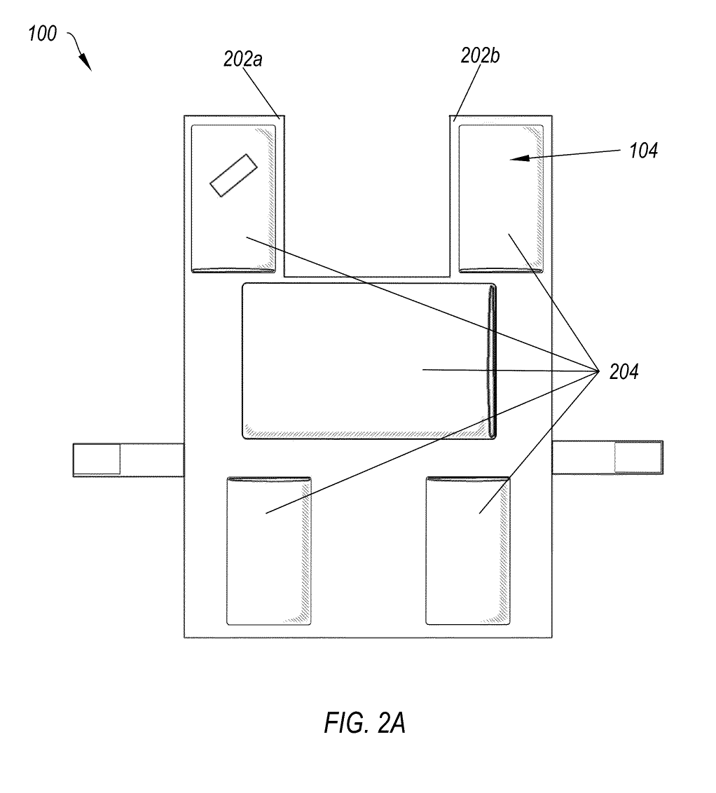

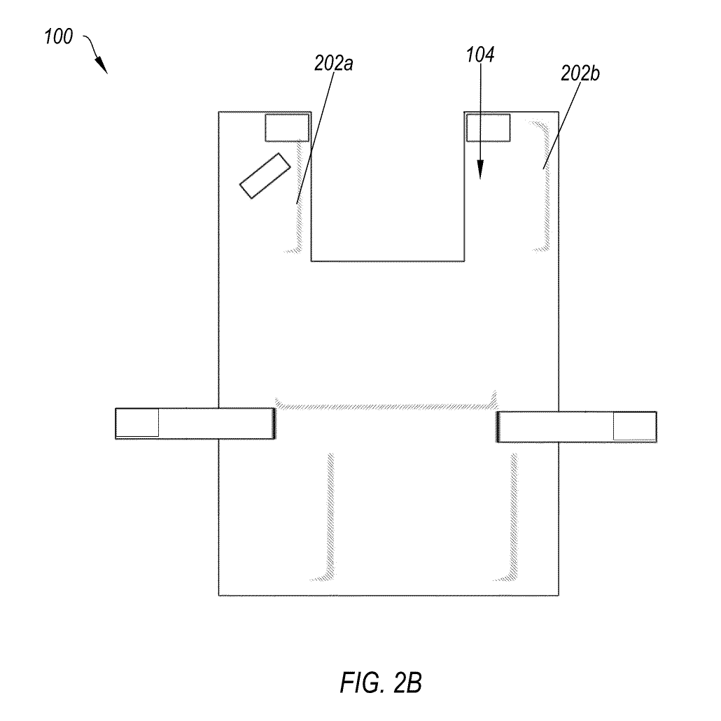

FIGS. 2A and 2B illustrate a view of the ice harness 100 when not in use. FIG. 2A is an “inside” view of the ice harness 100; and FIG. 2B is an “outside” view of the ice harness 100. In at least one implementation, the ice harness 100 can include attachable parts, allow the user to assemble the ice harness 100 as needed. Additionally or alternatively, the ice harness 100 can allow the user to adjust or configure the ice harness 100 as desired.

FIGS. 2A and 2B show that the front section 104 can include a first arm 202a and a second arm 202b (collectively “arms 202”). The arms 202 can allow a user to properly position the front section 104. In particular, the arms 202 can allow the user to wear the ice harness 100 without cooling his/her head. For example, the user can cross the arms 202 over one another, covering the user's chest without making contact with the user's neck or head.

FIGS. 2A and 2B also show that the ice harness 100 can include one or more cold-gel pack pockets 204. In at least one implementation, the cold-gel pack pockets 204 can be configured to receive and hold a cold-gel pack in the ice harness 100. I.e., the user can freeze or otherwise cool the cold-gel pack and then attach it to the ice harness 100 using the cold-gel pack pockets 204. One of skill in the art will appreciate that the ice harness 100 need not be placed in a pocket 204 but can be attached or placed in the desired area using other means. I.e., any mechanism for placing the ice pack in the desired location is contemplated herein. For example, the cold-gel pockets 204 can be located in the first arm 202a and the second arm 202b. Additionally or alternatively, the cold-gel pockets 204 can be located along the user's upper back or lower back, as desired.

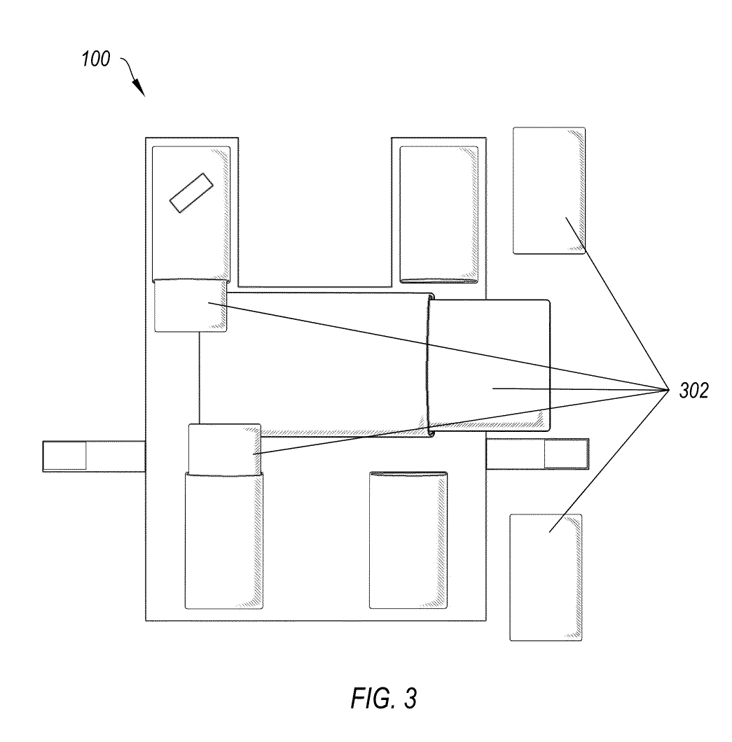

FIG. 3 illustrates an exploded view of the ice harness 100 with some of the cold-gel packs 302 partially placed within the pockets 204. In at least one implementation, the cold-gel packs 302 can be approximately the same size and shape as the tissue to which the cold treatment will be applied. I.e., the cold-gel packs 302 can cover all or substantially all of the desired tissue areas when worn by the user.

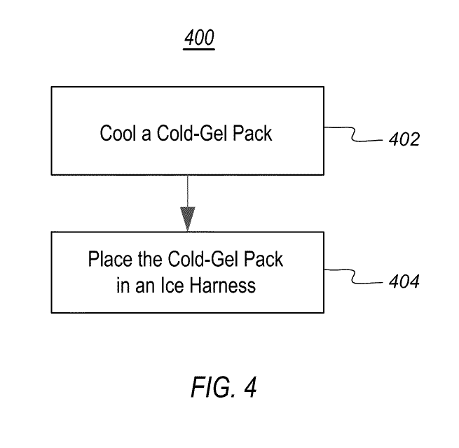

FIG. 4 is a flow chart illustrating a method 400 of cooling brown adipose tissue. One of skill in the art will appreciate that the method 400 can be used with the ice harness 100 of FIGS. 1-3; however, the method 400 can be used with a ice harness other than the ice harness 100 of FIGS. 1-3.

FIG. 4 shows that the method 400 can include cooling 402 a cold-gel pack. In at least one implementation, cooling 402 a cold-gel pack can include placing the cold-gel pack in a freezer, fridge or other cooling device. In particular, the cold-gel pack can be placed in any device intended to remove heat from the cold-gel pack. Additionally or alternatively, the cold-gel pack can be cooled 402 by bringing it into contact with material that is colder that the cold-gel pack, such as ice, snow, dry ice or any other material.

FIG. 4 also shows that the method 400 can include placing 404 the cold-gel pack in an ice harness. In at least one implementation, the cold-gel pack can be left near brown adipose tissue for sufficient time to cool the brown adipose tissue. For example, the cold-gel pack can be placed near the brown adipose tissue for at least 20 minutes. E.g., the cold-gel pack can be placed near the brown adipose tissue between 30 and 45 minutes.

One of skill in the art will appreciate that the intensity and duration of cooling can affect the results seen by the user. For example, “casual” use can include general coolness, feeling generally “cooled off” which provides a mild calorie burn which adds up over time. In contrast, “beginner” use can include chilled spots, up to mild goose bumps which can provide calorie burn which may burn off glucose from bloodstream. Moreover, “moderate” use can include strong goose bumps, up to a slight muscle-twitch which can provide skeletal muscles uncoupling, BAT activation, significant calorie burn, glucose clearing, and boosted metabolism. Additionally, “hardcore” use can include pre-shiver to full shivering which may include Adiponectin increases, direct fat burn and massive calorie burn from shivering.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An ice harness for cooling brown adipose tissue, the ice harness comprising:
a front section, wherein the front section is configured to be placed on a user's chest;
a rear section, wherein the rear section is configured to be placed on the user's upper back;
a first cold-gel pack, wherein the first cold-gel pack is attached to the front section; and
a second cold-gel pack, wherein the second cold-gel pack is attached to the rear section.

2. The system of claim 1, wherein the front section includes a first arm.

3. The system of claim 2, wherein the front section includes a second arm.

4. The system of claim 3, wherein the first arm and the second arm are configured to cross over one another during use.

5. The system of claim 3, wherein the first arm includes a pocket.

6. The system of claim 5, wherein the first arm is configured to locate the first cold-gel pack on the user's chest.

7. The system of claim 6, wherein the second arm includes a pocket.

8. The system of claim 7, wherein the second arm is configured to locate the second cold-gel pack on the user's chest.

9. The system of claim 1, wherein the back section includes a pocket.

10. The system of claim 9, wherein the pocket is configured to locate a cold-gel pack on the user's upper back.

11. The system of claim 10, wherein the back section includes a second pocket.

12. The system of claim 11, wherein the second pocket is configured to locate a cold-gel pack on the user's lower back.

13. The system of claim 12, wherein the back section includes a third pocket.

14. The system of claim 13, wherein the third pocket is configured to locate a cold-gel pack on the user's lower back adjacent to the second pocket.

15. An ice harness for cooling brown adipose tissue, the ice harness comprising:
a front section, wherein the front section is configured to be placed on a user's chest;
a rear section, wherein the rear section is configured to be placed on the user's upper back;
a first cold-gel pack, wherein the first cold-gel pack is attached to the front section;
a second cold-gel pack, wherein the second cold-gel pack is attached to the rear section;
a shoulder support, wherein the shoulder support is configured to secure the front section and the rear section vertically relative to the user; and
an under-arm support, wherein the under-arm support is configured to secure the front section and the rear section horizontally relative to the user.

16. The system of claim 15, wherein the under-arm support includes a strap.

17. The system of claim 16, wherein the strap includes a hook and loop fastener.

18. A method for cooling brown adipose tissue, the method comprising:
cooling a cold-gel pack; and
placing the cold-gel pack in an ice harness, wherein the ice harness includes:
a front section, wherein the front section is configured to be placed on a user's chest; and
a rear section, wherein the rear section is configured to be placed on the user's upper back.

19. The system of claim 18, wherein the cold-gel pack is attached to the front section.

20. The system of claim 18, wherein the cold-gel pack is attached to the rear section.
